(19) United States
(12) Patent Application Publication
(10) Pub. No.: US 2019/0099490 A1
(43) Pub. Date: Apr. 4, 2019

(54) APPARATUS AND METHOD FOR MAGNETIC DELIVERY OF DRUGS DEEP INTO ARTICULAR CARTILAGE FOR OSTEOARTHRITIS

(71) Applicant: Weinberg Medical Physics, Inc., North Bethesda, MD (US)

(72) Inventors: Irving N. WEINBERG, North Bethesda, MD (US); Sahar JAFARI, North Bethesda, MD (US); Lamar Odell MAIR, Baltimore, MD (US)

(21) Appl. No.: 16/151,977

(22) Filed: Oct. 4, 2018

Related U.S. Application Data
(60) Provisional application No. 62/567,871, filed on Oct. 4, 2017.

(51) Int. Cl. A61K 41/00; A61B 5/055; A61K 9/00; A61K 31/56; A61K 38/18; A61K 31/7052; A61K 9/14

(52) U.S. Cl. CPC A61K 41/00; A61B 5/055; A61K 9/0019; A61B 34/73; A61K 38/18; A61K 31/7052; A61K 9/14; A61K 31/56

(57) ABSTRACT

Disclosed embodiments provide a tool and methodologies for delivering drugs into articular cartilage of a subject's body.

### CROSS REFERENCE AND PRIORITY CLAIM

This patent application claims priority to U.S. Provisional Application Provisional Patent Application No. Patent Application Ser. No. 62/567,871, entitled “APPARATUS AND METHOD FOR MAGNETIC DELIVERY OF DRUGS DEEP INTO ARTICULAR CARTILAGE FOR OSTEOARTHRITIS,” filed Oct. 4, 2017, the disclosure of which being incorporated herein by reference in its entirety.

### FIELD

Disclosed embodiments provide a tool for delivering drugs into articular cartilage of subject's body.

### BACKGROUND

Sometimes called degenerative joint disease or degenerative arthritis, Osteoarthritis (OA) is the most common chronic (long term) condition of the joints, affecting approximately 27 million American humans. Although OA occurs in people of all ages, osteoarthritis is most common in people older than 60. About 10% of men and 18% of women over 60 years of age have OA.

In normal human joints, a firm, rubbery material called cartilage covers the end of each bone. In normal use, cartilage provides a smooth, gliding surface for joint motion and acts as a cushion between the bones. However, in OA, the cartilage breaks down, causing pain, swelling and problems moving the joint.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Disclosed embodiments provide a tool and methodologies for delivering drugs into articular cartilage of a subject's body.

Disclosed embodiments provide a non-invasive method and associated equipment for delivery of drugs into articular cartilage of a subject's body.

In accordance with disclosed embodiments, one or more magnetizable particles may be introduced non-invasively into one or more body structures within a subject.

In accordance with disclosed embodiments, at least one image-guidance component located in proximity to the one or more body structures may be used to direct, transport, concentrate and/or focus the one or more particles within the one or more body structures within a subject.

### BRIEF DESCRIPTION OF FIGURES

Further advantages, features and possibilities of using the present disclosed embodiments emerge from the description below in conjunction with the figures.

FIG. 1 illustrates the principle of the disclosed innovation wherein a test apparatus contains a solution of magnetic nanoparticles retained within a volume by an O-ring.

FIG. 2 illustrates an embodiment of the apparatus in which a single-sided magnetic resonance instrument uses magnetic fields to image a subject's joint.

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Unfortunately, there is no cure for OA, but conventional treatments are available to manage symptoms. Research on development of disease-modifying OA drugs has been active in recent decades. Several conventionally known drugs have potential to inhibit cartilage degeneration associated with OA and to promote cartilage repair; however, none of these drugs have yet translated to clinical practice, due to the lack of effective delivery systems that enable local, safe administration in low doses without off-target effects.

OA lesions in articular cartilage can be very localized. Thus, local administration of the drug could potentially enhance the therapeutic effect within the degenerated tissue while avoiding adverse effects elsewhere in the body.

Non-destructive localized delivery of drugs into articular cartilage could, therefore, lead to new treatment strategies for OA therapy. However, drugs need to penetrate as deep as possible within cartilage to reach the chondrocytes and ExtraCellular Matrix (ECM) targets involved in OA-associated cartilage pathogenesis before the drugs are cleared from the joint by physiological processes (e.g., circulation, enzymatic degradation, absorption by non-cartilage components of the joint).

Another conventional obstacle to effective drug delivery is the small pore size in dense joint cartilage, which has stymied attempted experimental methods of increasing the depth of delivery (e.g., with electric charge; see Ambika G. Bajpayee and Alan J. Grodzinsky, ‘Cartilage-Targeting Drug Delivery: Can Electrostatic Interactions Help?’, Nature Reviews Rheumatology, 13.3 (2017), 183-93, incorporated herein in its entirety), ultrasound (H. J. Nieminen and others, ‘Ultrasonic Transport of Particles into Articular Cartilage and Subchondral Bone’, IEEE International Ultrasonics Symposium, IUS, 2012, 1869-72), incorporated herein in its entirety, and acoustic shock waves (Heikki J Nieminen and others, ‘MHz Ultrasonic Drive-in: Localized Drug Delivery for Osteoarthritis Therapy’, IEEE International Ultrasonics Symposium, IUS, 2013, 619-22, incorporated herein in its entirety).

However, conventional methods mentioned above have never been successful in delivering drugs to the entire thickness of cartilage.

To the contrary, in accordance with the disclosed embodiments, an apparatus and method are provided that enable the ability to propel drug-loaded or non-drug-loaded magnetic particles through some or all of a human subject's cartilaginous tissue. The invention consists of an apparatus and method which can be used to propel drug-loaded or non-drug-loaded magnetic particles through some or all of a cartilaginous tissue. One or more coils placed near the tissue (e.g., up to one meter away) generate an oscillating magnetic field to wiggle the nanoparticles. It is understood that the terms “coil” or “coils” includes coils surrounding or in the vicinity of magnetizable material, for example as in electropermanent magnets. The use of electropermanent magnets in magnetic resonance imaging and magnetic particle delivery was described by Irving Weinberg in U.S. Provisional patent 62/688,568, entitled “METHOD FOR ACQUIRING AN IMAGE AND MANIPULATING OBJECTS WITH MAGNETIC GRADIENTS PRODUCED BY ONE OR MORE ELECTROPERMANENT MAGNET ARRAYS”, and incorporated by reference.

The technical effect of the disclosed embodiments provides the ability to improve the efficacy of drugs in changing the natural course of joint disease (from injury to degenerative arthritis) by enabling delivery of the drugs through a substantial portion, (e.g., more than a few, for example, more than 100 microns) of the entire thickness of articular cartilage, for example, a majority of the entire thickness of articular cartilage, rapidly enough to prevent diffusion of the drugs from the cartilage. Thus, a beneficial effect of using this apparatus is to deliver medications deep into cartilage extracellular matrix with drug-loaded magnetic particles propelled with real-time shaped dynamic magnetic fields.

Under the guidance of oscillating magnetic fields, the magnetic nanoparticles may drive their drug payloads deep into cartilage faster than published clearance times for these drugs. Example of potential useful drugs that might be loaded onto the magnetic particles include steroids, drugs affecting anabolic signaling pathways (TGF-β), and IL-1 receptor antagonists.

For the purposes of this disclosure, the terms “magnetic particles” and “nanoparticles” and “magnetic nanoparticles” are defined as one or more particles that can be magnetized using magnetic fields, where the maximum dimension of the particle is one millimeter or less.

The term “oscillating magnetic field” is defined as alternating magnetic fields provided by Helmholtz coils. For the purposes of this specification, the term “oscillating” means changing in direction and/or magnitude. The term “static magnetic field” is defined as a magnetic field which does not change in intensity or direction over time. In this specification, the term “cartilage” and “cartilaginous tissue” refer to all forms of cartilage and joint surfaces. For the purposes of this disclosure, the terms “superficial cartilage surface” is defined as the surface of cartilage in contact with magnetic nanoparticles and the term “deep cartilage surface” is defined as the surface of cartilage which is the furthest from magnetic nanoparticles, respectively. For the purposes of this disclosure, the term “drug-loaded particles” means particles bound to, covered by, or incorporating or carrying a therapeutic compound or substance (e.g., steroid, growth factor, nucleic acid).

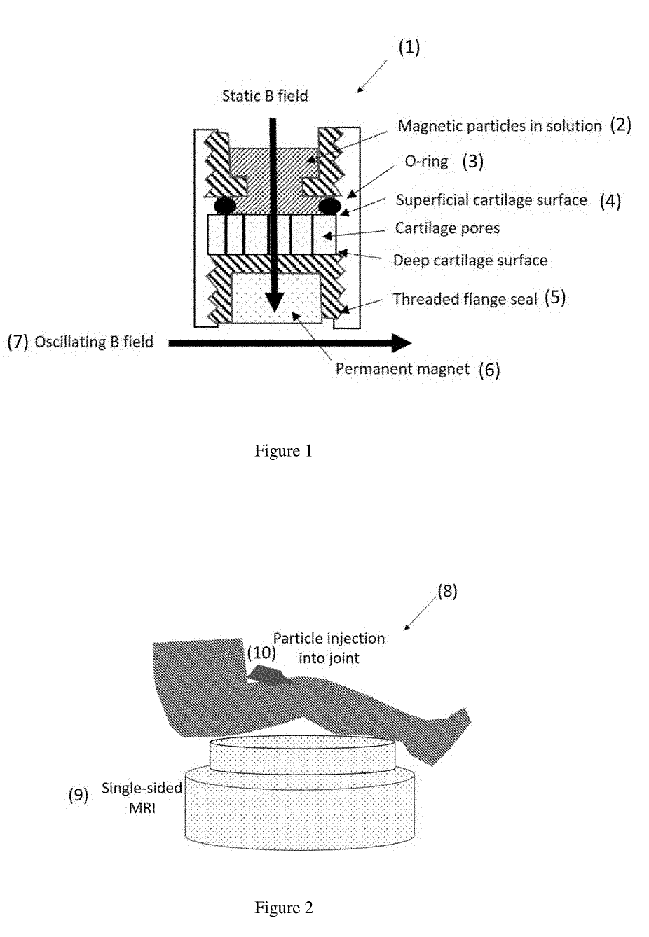

FIG. 1 illustrates the principle of the disclosed embodiments, wherein a permanent magnet applies a static field to nanoparticles, and a set of coils is used to apply an oscillating magnetic field. As shown in FIG. 1, a test apparatus 1 contains a solution of magnetic nanoparticles 2 retained within a volume by an O-ring 3. The solution 2 is in contact with a cartilage surface 4. The test apparatus includes a threaded flange seal 5, permanent magnet 6, and a coil to produce an oscillating magnetic field 7.

An embodiment of the apparatus as applied to therapy is shown in FIG. 2. FIG. 2 illustrates an embodiment of the apparatus 8 in which a single-sided magnetic resonance instrument 9 uses magnetic fields to image the joint. In accordance with disclosed embodiments, a solution containing drug-loaded magnetic nanoparticles 10 may be injected into a subject's joint. The drug-loaded particles may be administered intra-venously to a patient and guided to a cartilaginous joint surface at least in part by a magnetic field applied by one or more coils external to the patient. Alternately, the drug-loaded particles may be administered via injection into the joint.

In accordance with various embodiments, magnetic gradients and fields may be generated by the magnetic resonance instrument 9 may also be used to propel drug-loaded magnetic particles through the cartilage surface. The magnetic resonance instrument 9 may alternately image and propel the drug-loaded magnetic particles, as previously disclosed in patent applications by Irving Weinberg. Thus, the magnetic resonance instrument 9 may include a system to apply magnetic fields under imaging guidance. Thus, image-guidance components may include permanent magnets, electromagnets, antennas or electropermanent magnets, as taught in Irving Weinberg in US Pat. Pub. 20170227617, corresponding to U.S. patent application Ser. No. 15/427,426, entitled “METHOD AND APPARATUS FOR MANIPULATING ELECTROPERMANENT MAGNETS FOR MAGNETIC RESONANCE IMAGING AND IMAGE GUIDED THERAPY,” incorporated herein by reference. Such electropermanent magnets may at one or more times create a magnetic field configuration for imaging of a subject's body part and then at another set of times create a magnetic field configuration for propulsion of particles. It should be understood that the imaging capability may be through magnetic resonance imaging methods.

It should be understood that the disclosed apparatus and methodologies may be used in conjunction with other components, for example a computer and/or a power supply and/or coils for generating magnetic and/or electromagnetic fields, in order to attain a desired result of a meaningful image. It is understood that the image may use principles of proton magnetic resonance imaging, or magnetic resonance imaging of other particles (for example, electrons or sodium atoms) or other imaging principles (for example, magnetic particle imaging, or impedance imaging). It is understood that the apparatus may be used to deliver therapy by manipulating magnetizable materials with the magnetic field produced by the device. It should be understood that this manipulation may be performed at one time, and that imaging may be performed at another time, in order to guide the manipulation described above.

For the purpose of the disclosed embodiments, the term imaging, includes imaging technology that utilize components to form an image using magnetic resonance or magnetic particle imaging. It should be understood that such components include coils or magnets (or electro-permanent magnets) that polarize protons or other nuclei or electrons in one or more structures to be imaged, wherein gradient and/or radiofrequency coils form an image. Thus, although not shown in detail herein, it should be understood that the disclosed embodiments may be used in conjunction with a support structure that may hold an imaging system and may contain other components needed to operate or move the imaging system, for example, wheels and/or batteries. Moreover, it should be understood that an associated display system is not shown but should be understood to be present in order to view images produced by the imaging system.

It should be understood that one or more magnetic fields applied by the magnetic resonance instrument 9 to a body part of a subject may be so rapidly applied so as not to cause unpleasant nerve stimulation, as taught by Irving Weinberg in issued U.S. Pat. No. 8,154,286, entitled “APPARATUS AND METHOD FOR DECREASING BIO-EFFECTS OF MAGNETIC FIELDS” and related applications related through priority rights by Irving Weinberg, incorporated herein by reference.

With the above description in mind, it should be understood that the term “subject” refers to and includes humans and other animals, whether they be alive or once-living. Similarly, the term “body part or other structure” may mean a tissue-containing structure in a living or once-living organism such as a human or other animal.

Likewise, it should be understood that the term “structure” may mean a tissue-containing structure in a living or once-living organism such as a human or other non-human animal.

It should be understood that the term “magnetizable” and “magnetic” are used interchangeably to indicate a material that can be magnetized.

It should be understood that the term “magnetizable particle” may refer to a particle made of material that exhibits magnetic or electric properties after or during exposure to a magnetic field. It should be understood that the term “particle” means an object smaller than 1 mm, 100 micron, 10 microns, 1 micron, 0.1 microns, or 0.01 microns in the smallest diameter.

The terms “near” and “proximity” may be less than one meter.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term non-transitory is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While certain illustrative embodiments have been described, it is evident that many alternatives, modifications, permutations and variations will become apparent to those skilled in the art in light of the foregoing description. Accordingly, the various embodiments of, as set forth above, are intended to be illustrative, not limiting. Various changes may be made without departing from the spirit and scope of the invention.

1. An apparatus for delivering a plurality of drug-loaded magnetic particles to a substantial amount of an entire thickness of cartilage of a cartilaginous joint of a subject, the apparatus comprising:
one or more coils disposed near to the cartilage and outside a body of the subject;
at least one image-guidance component positioned in proximity to the subject's cartilage; and
a controller coupled to the one or more coils and configured to control the one or more coils to generate an oscillating magnetic field,
wherein the at least one image-guidance component provides imaging data that enables delivery of the plurality of drug-loaded magnetic particles to a substantial amount of the entire thickness of the cartilage of the subject's cartilaginous joint.

2. The apparatus of claim 1, further comprising the plurality of drug-loaded magnetic particles, which are loaded with a least one of a steroid, a drug affecting anabolic signaling pathways), an IL-1 receptor antagonists, a growth factor and a nucleic acid.

3. The apparatus of claim 1, wherein the one or more coils are Helmholtz coils.

4. The apparatus of claim 1, wherein the plurality of drug-loaded magnetic particles are injected into a joint of the subject.

5. The apparatus of claim 1, wherein the plurality of drug-loaded magnetic particles are administered intra-venously to the subject and guided to a cartilaginous joint surface at least in part by a magnetic field applied by one or more coils external to the patient.

6. The apparatus of claim 1, wherein the at least one image-guidance component comprises an MRI system that includes the at least one magnetic coil.

7. The apparatus of claim 6, wherein the MRI system is a single-sided MRI system.

8. The apparatus of claim 6, wherein the MRI system includes a plurality of electropermanent sections.

9. The apparatus of claim 6, wherein the MRI system generates pulse sequences wherein the MRI has pulse sequences whose rise-time, fall-time, or duration are less than 10 microseconds long.

10. The apparatus of claim 6, wherein the MRI system includes at least one magnetic coil generates a magnetic field that rises or falls in such short a time as not to cause nerve stimulation of the subject.

11. The apparatus of claim 9, wherein the at least one magnetic coil generates a magnetic field that rises or falls in less than 10 microseconds.

12. A method of delivering drugs to a subject's cartilaginous joint, the method comprising:
administering a plurality of drug-loaded magnetic particles to the subject; and
performing image-guided magnetic delivery of the one or more drug-loaded magnetic particles to the subject's cartilaginous joint by propelling the plurality of drug-loaded magnetic particles into a substantial amount of an entire thickness of cartilage of the cartilaginous joint of the subject at least in part by applying an oscillating magnetic field to the cartilage using one or more coils.

13. The method of claim 12, wherein the plurality of drug-loaded magnetic particles are loaded with a least one of a steroid, a drug affecting anabolic signaling pathways), an IL-1 receptor antagonists, a growth factor and a nucleic acid.

14. The method of claim 12, wherein the one or more coils are Helmholtz coils.

15. The apparatus of claim 12, wherein the plurality of drug-loaded magnetic particles are injected into a joint of the subject.

16. The method of claim 12, wherein the plurality of drug-loaded magnetic particles are administered intra-venously to the subject and guided to a cartilaginous joint surface at least in part by a magnetic field applied by one or more coils external to the patient.

17. The method of claim 12, wherein the at least one image-guidance component comprises an MRI system that includes the at least one magnetic coil.

18. The method of claim 17, wherein the MRI system is a single-sided MRI system.

19. The method of claim 17, wherein the MRI system includes a plurality of electropermanent sections.

20. The method of claim 17, wherein the MRI system generates pulse sequences wherein the MRI has pulse sequences whose rise-time, fall-time, or duration are less than 10 microseconds long.

21. The method of claim 17, wherein the MRI system includes at least one magnetic coil generates a magnetic field that rises or falls in such short a time as not to cause nerve stimulation of the subject.

22. The method of claim 20, wherein the at least one magnetic coil generates a magnetic field that rises or falls in less than 10 microseconds.
